SEQUENCE LISTING (6 sequences)

SEQ ID NO: 1 - protein, 4 aa, Unknown
  Description of Unknown ′DEAD′ box domain peptide
DEAD

SEQ ID NO: 2 - protein, 4 aa, Unknown
  Description of Unknown ′DEAH′ box domain peptide
DEAH

SEQ ID NO: 3 - protein, 9 aa, Unknown
  Description of Unknown ′LAGLIDADG′ family peptide
LAGLIDADG

SEQ ID NO: 4 - DNA, 67 bp, Artificial Sequence
  Description of Artificial Sequence Synthetic oligonucleotide
cttttcacactgtatcccaatgctgccatcattgcaaagattgcggactacggcattgctcagtact

SEQ ID NO: 5 - DNA, 67 bp, Unknown
  Description of Unknown Wild-type leucine rich repeat kinase 2 oligonucleotide
cttttcacactgtatcccaatgctgccatcattgcaaagattgctgactacggcattgctcagtact

SEQ ID NO: 6 - DNA, 67 bp, Artificial Sequence
  Description of Artificial Sequence Synthetic oligonucleotide
cttttcacactgtatcccaatgctgccatcattgcaaagattgctgactacagcattgctcagtact

(19) United States
(12) Patent Application Publication
(10) Pub. No.: US 2016/0010154 A1
(43) Pub. Date: Jan. 14, 2016

(54) SCREENING ASSAYS FOR THERAPEUTICS FOR PARKINSON'S DISEASE

(71) Applicants: THE PARKINSON'S INSTITUTE, Sunnyvale, CA (US); SANGAMO BIOSCIENCES, INC., Richmond, CA (US)

(72) Inventors: Josee LAGANIERE, Richmond, CA (US); Birgitt SCHÜLE, Menlo Park, CA (US)

(21) Appl. No.: 14/647,732

(22) PCT Filed: Dec. 2, 2013
(86) PCT No.: PCT/US13/72701

§ 371 Date: May 27, 2015

Related U.S. Application Data
(60) Provisional application No. 61/731,748, filed on Nov. 30, 2012.

(51) Int. Cl. C12Q 1/68

(52) U.S. Cl. CPC C12Q 1/6883; C12Q 2600/106; C12Q 2600/118

(57) ABSTRACT

Disclosed herein are in vitro and in vivo methods for screening for compounds that treat diseases and conditions that are related to oxidative stress such as Parkinson's disease

### CROSS-REFERENCE

This application claims priority to U.S. Provisional Patent Appl. No. 61/731,748 filed on Nov. 30, 2012, the contents of which is incorporated by reference in its entirety.

### BACKGROUND OF THE INVENTION

Parkinson's disease (PD) is a neurodegenerative disease that afflicts approximately 4-6 million people worldwide. In the United States, approximately one to two hundred people per 100,000 have PD. Interestingly, the prevalence among Amish people is approximately 970 per 100,000 although the basis for this high rate, be it genetic or environmental, is not known. The prevalence of PD increases in the older population, with approximately 4% of people over the age of 80 suffering from this disease (Davie (2008) Brit Med Bull 86(1) p. 109), although 10% of patients are under 40 years of age (Kumari (2009) FEBS J. 276(22) p. 6455).

Typically a patient diagnosed with PD is identified by several hallmark physical behaviors: bradykinesia, rigidity and resting tremor. Often these physical symptoms are asymmetric. Within the brain, PD is characterized by a progressive and profound loss of neuromelanin-containing dopaminergic neurons in the substantia nigra pars compacta with the presence of eosinophillic, intracytoplasmic and proteinaceous inclusions termed Lewy bodies in the surviving neurons (Davie, ibid and Kumari, ibid). By the time of death, this region will have lost 50-70% of its neurons as compared to an individual without PD.

It appears that many factors can play a role in disease onset and/or progression of PD. For example, genetic mutations in the leucine rich repeat kinase 2 gene (LRRK2, also known as PARK8) have been identified to be involved in both familial and sporatic forms of PD. See, e.g., U.S. Patent Publication No. 20120192301. For example, G2019S has been suggested to play an important role in PD in some ethnicities. (Luzon-Toro, (2007) Hum Mol Genet 16(17) p. 2031).

Currently, it is difficult to screen for PD therapeutics, in part, due to the difficulty in obtaining in vitro models for PD (e.g., isogenic lines for high-throughput screening). Schüle et al. (2009) Biochimica et biophysica acta 1792: 1043-1051 (2009); Hartfield et al. (2012) Biochemical Society transactions 40:1152-1157 (2012). In PD, reactive oxygen species (ROS) damage lipids and proteins (Sherer (2005) Antioxid Redox Signal 7:627) but less is known about damage to mitochondrial DNA (mtDNA) (Sanders et al. Free Radic Biol Med (In press). DNA damage is defined as any modification of DNA that can alter its coding properties or can interfere with normal function in transcription or replication. Lindahl, (1993) Nature 362:709-15 (1993); Rao (1993) Mol Neurobiol 7, 23-48. The mitochondrial genome is particularly susceptible to oxidative damage, likely due to the proximity of mtDNA to ROS production at the inner mitochondrial membrane and the lack of protection afforded by histones. Yakes et al. (1997) Proc Natl Acad Sci USA 94: 514-9. Mitochondrial DNA damage can compromise metabolic functions, predispose to ROS generation and trigger cell death. The accumulation of mtDNA damage is a particular problem for the brain since neurons are post-mitotic and long-lived and damage to mtDNA may lead to mtDNA mutations.

Thus, there remains a need for the development of novel in vitro models of PD, including in vitro models for screening PD therapeutics, for example based on the effect on mitochondrial DNA.

### SUMMARY OF THE INVENTION

Disclosed herein are methods and compositions for the development of in vitro and in vivo systems for evaluation of PD, including high throughput screening of PD therapeutics.

In one aspect, provided herein is an isogenic cell line comprising wild-type or mutant LRRK2 alleles. The cell line may comprise one or more mutations at LRRK2, for example, a G2019S mutation. The cell line may be iPSCs (e.g., patient-derived iPSCs). The isogenic cells lines are preferably prepared using a nuclease (e.g., a zinc finger nuclease), which modifies LRRK2 via homology or non-homology mediated repair.

In another aspect, described herein is an organism comprising wild-type or mutant LRRK2 alleles. The organism may comprise one or more mutations at LRRK2, for example, a G2019S mutation. The organism may be a non-human mammal (e.g., mouse, rat, rabbit, etc.). The organisms are preferably prepared using a nuclease (e.g., a zinc finger nuclease), which modifies LRRK2 via homology or non-homology mediated repair in a cell and the cell is allowed to develop into the organism.

In another aspect, described herein is a method of preparing an isogenic cell line, the method comprising using one or more nucleases (e.g., ZFNs) to modify an endogenous LRRK2 gene in the cell. The nuclease cleaves the endogenous LRRK2 gene and modification occurs via homology (targeted integration of a donor polynucleotide) or non-homology (NHEJ) mechanisms. In certain embodiments, the cell line comprises iPSCs (e.g., patient-derived iPSCs). The methods described herein provide selection-free, sorting-free isolation of cells carrying investigator-specified LRRK2 alleles. Furthermore, LRRK2-nuclease modified iPSCs maintain their stemness, normal karyotype and potential for neuronal differentiation.

In yet another aspect, provided herein is a method of screening for a compound useful in the treatment of Parkinson's disease or Parkinson's-related disease, the method comprising: providing an isogenic cell line or organism with a wild-type or modified LRRK2 allele as described herein; and assaying the isogenic cell line or organism for a response to the compound. For example, production of free radicals is measured, wherein if the compound reduces the amount of free radicals in the cell line or organism as compared to a control cell or organism not receiving the compound, the compound is identified as one useful in the treatment of Parkinson's disease or Parkinson's-related disease. In certain embodiments, the conditions of oxidative stress are selected from the group consisting of nutritional challenge (e.g., nutrient withdrawal), challenge with toxins (e.g., rotenone) and combinations thereof.

In another aspect, provided herein is a method for assaying the effect of a compound on mitochondrial membrane potential (MMP) the method comprising: providing an isogenic cell line or organism with a wild-type or modified LRRK2 allele as described herein; administering the compound to the isogenic cell line or organism under conditions of oxidative stress on the cell or organism, assaying MMP of the cell line or organism, wherein if the compound increases MMP as compared to a control cell or organism not receiving the compound, the compound is identified as one that increases MMP. In certain embodiments, the conditions of oxidative stress are selected from the group consisting of nutritional challenge, challenge with toxins (e.g., rotenone, staurosporine) and combinations thereof.

In a still further aspect, described herein is a method for assaying the effect of a compound on mitochondrial transitional pore opening (MTP), the method comprising: providing an isogenic cell line or organism with a wild-type or modified LRRK2 allele as described herein; administering the compound to the isogenic cell line or organism under conditions of oxidative stress on the cell or organism, assaying MTP of the cell line or organism (e.g., by loading with a calcium chelator such as calcein), wherein if the compound increases MTP as compared to a control cell or organism not receiving the compound, the compound is identified as one that increases MTP. In certain embodiments, the conditions of oxidative stress are selected from the group consisting of nutritional challenge, challenge with toxins (e.g., rotenone, staurosporine) and combinations thereof.

In a still further aspect, described herein is a method of evaluating the effect of a compound for treating of PD, the method comprising: providing an providing an isogenic cell line or organism with a wild-type or modified LRRK2 allele as described herein; administering the compound to the isogenic cell line or organism, assaying the cell line or organism for one or more of the following: production of free radicals in response to oxidative stress, MMP, MTP, caspase activation or combinations thereof, wherein a reduction in the amount of free radicals, an increase in MMP, an increase in MTP or a decrease in caspase activity (as compared to a control cell or organism) not receiving the compound) is indicative of a compound that treats PD.

In a further aspect, provided is a method of determining whether a compound is useful in the treatment of Parkinson's disease or Parkinson's-related disease, the method comprising: (a) providing an isogenic cell line comprising a modified LRRK2 allele; (b) contacting the isogenic cell line with the compound; and (c) assaying the isogenic cell line for a reduction of mitochondrial DNA damage or a reduction in the rate of mitochondrial DNA damage, thereby determining whether the agent is useful in the treatment of Parkinson's disease or Parkinson's-related disease.

These and other aspects will be readily apparent to the skilled artisan in light of disclosure as a whole.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1, panels A to D, show ZFN targeting of LRRK2. FIG. 1A is a schematic illustration of the ZFN targeting sequence as well as restriction enzymes sites present in wild-type, G6055A mutation, and donor DNA used for clone screening. The BsrDI site lies directly between the two ZFNs right at the cleavage site and a loss of this site will indicate a NHEJ event; the SfcI enzyme cleaves the mutated DNA sequence exclusively, therefore a loss of this site is necessary to confirm gene correction; and the AciI is the silent RFLP engineered within the donor of wild type sequence, which will co-convert with the wild type donor DNA in the event of homologous recombination/gene correction. FIG. 1B shows an RFLP assay of the LRRK2 G2019S iPSCs nucleofected with wt or mutation-specifying ZFN, along with AciI+ wild type plasmid donor DNA. Co-delivery of LRRK2-specific ZFN encoding mRNA and donor plasmid showed 8.4% donor-specific integration detected by novel AciI silent RFLP. FIG. 1C is a schematic showing the derivation and genotype screening of the clones derived from ZFN-modified iPSCs. FIG. 1D shows exemplary results following AciI and BsrDI digests of 18 iPSC clones. Genome editing scheme approach: loss of Bsdr1 site indicates allele disruption, AciI site is engineered in donor sequence and indicates integration, loss of LRRK2 p.G2019S SfcI site indicates mutant allele correction. Example for AciI and BsdrI digest (top and middle panels) with two positive clones 7 and 11 (clone numbers are indicated at the bottom of each lane in the figure). Same clones 7 and 11 also lost the SfcI site indicating that the donor is integrated on the mutant allele (bottom panel).

FIG. 2, panels A and G, depict characterization of ZFN clones for pluripotency and neuronal differentiation potential. FIG. 2A shows representative phase contrast image of iPSC colony morphology. FIG. 2B immunocytochemistry on colonies demonstrated the expression of further pluripotency associated markers. FIG. 2C shows neural stem cells stained for Nestin and Sox1; FIGS. 2D-2F show immunocytochemical analysis of embryoid body formation demonstrated the expression of all 3 germ layer markers. FIG. 2G shows G-banding of metaphase cells confirmed a normal male karyotype.

FIG. 3, panels A to L, depict neuronal differentiation and morphological differences between ZFN-corrected and LRRK2,p.G2019S mutant. FIG. 3A shows co-immunostain of beta-III-tubulin (green) and TH (red). FIG. 3B shows co-immunostain of MAP2 (red) and TH (green). FIG. 3C shows co-immunostain of VMAT (red) and TH (green). FIG. 3D shows co-immunostain of synapsin I (red) and TH (green). FIG. 3E shows co-immunostain of alpha-synuclein (green) and TH (red). FIG. 3F shows co-immunostain of GABA (red) and TH (green) G) co-immunostain TH/MAP2 stain used for HCI counting. FIG. 3H shows significant differences in the percentage of TH+ neurons; 1.7 corrected 6.001% TH positive cells±0.9701 (N=12), 1.13 mutant 2.725% TH positive cells±0.2637 (N=15) (p=0.0014). FIG. 3I shows a scheme for analysis in neurite outgrowth module (MetaXpress Software). FIGS. 3J to 3L are graphs showing neurite outgrowth is improved in ZFN-corrected LRRK2 neurons. Significant decreases in mean number of processes and neurite length (mm) per cell in the LRRK2, p.G2019S culture compared with the control. There was a trend, but no significant difference, in the mean number of branches per cell in the LRRK2, p.G2019S culture compared to control. The asterisk,* represents means±SEM (n=3).

FIG. 4, panels A to D, shows mitochondrial function is improved in ZFN-corrected LRRK2 neuroprogenitor cells. FIG. 4A shows ROS production was measured by flow cytometry in uncorrected (1.13) and corrected (1.7) NPCs. Cells grown on high glucose (HG) with or without 20 uM rotenone (R) or with radical generator TBHP (200 uM). Cells were analyzed by flow cytometry and mean CM-DCFDA fluorescence (Ex./Em. 495/529 nm) of live cells was then normalized to number of viable cells. FIG. 4B shows HCI microscopy of MMP measured by the integrated JC-10 dye fluorescence ratio of mitochondrial JC-10 J-aggregates (red fluorescence Em. 590 nm) versus the monomeric form of JC-10 (green fluorescence at Em. 525 nm) (bottom panels of FIG. 4B) as well as a graph (top panel) depicting integrated mitochondrial JC-10 fluorescence intensity ratios that were measured under different conditions such as high glucose (HG), staurosporine (SP), rotenone (R), and no glucose (NG) by acquisition of 4 sites in four replicate wells of a 96 well plate using ImageXpress automated microscope (20×) and analyzed using MetaXpress software (Molecular Devices, LLC). Images: Depicted are representative image overlays showing mitochondrial fluorescence patterns indicative of mitochondrial membrane potential for the corrected (1.7) and uncorrected (1.13) NPCs. FIG. 4C shows HCI microscopy analysis of mitochondrial integrity as measured by mitochondrial calcein retention in NPCs (bottom panels) and a graph showing relative amounts of mitochondrial localized calcein (top panel). Mitochondria were loaded with calcein AM and cytosolic fluorescence was quenched by CoCl2. Graph: Relative amount of mitochondrial localized calcein fluorescence identified by Mitotracker™ Red CMXROS co-localization was normalized to cell number. Images: Representative image overlays showing mitochondrial fluorescence patterns indicative of mitochondrial calcein retention and mitochondrial integrity in corrected (1.7) and uncorrected (1.13) NPCs. FIG. 4D is a graph showing HCI analysis of caspase 3/7 activation in LRRK2 NPCs measured by fluorescence activated by caspase 3/7 mediated cleavage of a DEVD peptide-DNA dye conjugate. NPCs were incubated with the caspase substrate and the % of caspase substrate fluorescence positive cells (identified by nuclear Hoechst 33342 counterstain) was determined from 4 sites acquired at 10× magnification.

FIG. 5, panels A and B, are graphs depicting mtDNA damage in neural cells. FIG. 5A shows neural cells that were differentiated from iPSCs derived from LRRK2 mutation carriers with the G2019S (black bars) and R1441C (gray bars) mutations and from healthy subjects (white bars). Mitochondrial DNA lesions were increased in neural cells from individual iPSC clones carrying LRRK2 mutations (L1-3 and L5-6) relative to neural cells from healthy subjects' iPSCs (C1-3). FIG. 5B shows results from parallel, neural cells from individuals carrying LRRK2 mutations (black and grey bars) contained a similar number of mtDNA copies as neural cells from healthy subjects. Data are presented as mean±SEM.

FIG. 6, panels A to F, depict genomic repair of the LRRK2 G2019S mutation reduced mtDNA damage in neuroprogenitor cells and neural cells. FIGS. 6A-D are representative images of immunocytochemistry show iPSC-derived NPCs that coexpressed nestin (green) and SOX1 (red) (FIGS. 6A and B) and neural cells that coexpressed β-III-tubulin (green) and tyrosine hydroxylase (TH, red) (FIGS. 6C and D). NPCs (FIG. 6E) and neural cells (FIG. 6F) differentiated from iPSCs that retained LRRK2 G2019S mutation after ZFN transfection (L4d^{Unmod}, black bar) exhibited greater levels of mtDNA damage than cells differentiated from ZFN-corrected iPSCs (L4b^{WT/WT}, white bar, *p<0.002). Data are presented as mean±SEM. Scale bar=200 μm.

FIG. 7 is a table showing details of individual iPSC clones.

### DETAILED DESCRIPTION OF THE INVENTION

Disclosed herein are compositions and methods for developing models and assays for PD therapeutics. In particular, site-specific genome editing (i.e., zinc finger nuclease-mediated cleavage and integration, TALEN or CRISPR) is used to create advanced models of Parkinson's Disease (PD) that provide novel tools for drug discovery, namely discovery of PD therapeutics. Thus, described herein are isogenic cell lines, e.g., isogenic panels of patient-derived induced pluripotent stem cells (iPSC), that carry different allelic forms at the endogenous genomic locus provides a powerful tool for assaying PD therapeutics.

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### DEFINITIONS

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

By “site-directed modifying polypeptide” or “RNA-binding site-directed polypeptide” or “RNA-binding site-directed modifying polypeptide” or “site-directed polypeptide” it is meant a polypeptide that binds RNA and is targeted to a specific DNA sequence. A site-directed modifying polypeptide as described herein is targeted to a specific DNA sequence by the RNA molecule to which it is bound. The RNA molecule comprises a sequence that is complementary to a target sequence within the target DNA, thus targeting the bound polypeptide to a specific location within the target DNA (the target sequence).

The binding sites for any site-specific genome editing systems such as Zinc finger, TALEN or CRISPR/Cas can be “engineered” to bind to a predetermined nucleotide sequence, for example via engineering (altering one or more amino acids) of the recognition helix region of a naturally occurring zinc finger, by engineering the RVDs of a TALEN protein or engineering the DNA-targeting segment of a subject DNA-targeting RNA of the CRISPR/Cas system. Therefore, engineered zinc finger proteins, TALENs or CRISPR/Cas are proteins that are non-naturally occurring. Non-limiting examples of methods for engineering zinc finger or TALEN proteins are design and selection. A designed zinc finger or TALEN protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536; WO 03/016496 and WO 2011/146121.

A “selected” zinc finger or TALEN protein or CRISPR/Cas is a protein not found in nature whose production results primarily from an empirical process such as phage display, interaction trap or hybrid selection. See e.g., U.S. Pat. No. 5,789,538; U.S. Pat. No. 5,925,523; U.S. Pat. No. 6,007,988; U.S. Pat. No. 6,013,453; U.S. Pat. No. 6,200,759; WO 95/19431; WO 96/06166; WO 98/53057; WO 98/54311; WO 00/27878; WO 01/60970; WO 01/88197; WO 02/099084 and WO 2011/146121.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to re-synthesize genetic information that will become part of the target, and/or related processes. Such specialized HR often results in an alteration of the sequence of the target molecule such that part or all of the sequence of the donor polynucleotide is incorporated into the target polynucleotide.

In the methods of the disclosure, one or more targeted nucleases as described herein create a double-stranded break in the target sequence (e.g., cellular chromatin) at a predetermined site, and a “donor” polynucleotide, having homology to the nucleotide sequence in the region of the break, can be introduced into the cell. The presence of the double-stranded break has been shown to facilitate integration of the donor sequence. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

In any of the methods described herein, additional pairs of zinc-finger or TALEN proteins or CRISPR/Cas can be used for additional double-stranded cleavage of additional target sites within the cell.

In certain embodiments of methods for targeted recombination and/or replacement and/or alteration of a sequence in a region of interest in cellular chromatin, a chromosomal sequence is altered by homologous recombination with an exogenous “donor” nucleotide sequence. Such homologous recombination is stimulated by the presence of a double-stranded break in cellular chromatin, if sequences homologous to the region of the break are present.

In any of the methods described herein, the first nucleotide sequence (the “donor sequence”) can contain sequences that are homologous, but not identical, to genomic sequences in the region of interest, thereby stimulating homologous recombination to insert a non-identical sequence in the region of interest. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In other embodiments, the donor sequence is non-homologous to the first sequence, and is inserted into the genome by non-homologous recombination mechanisms.

Any of the methods described herein can be used for partial or complete inactivation of one or more target sequences in a cell by targeted integration of donor sequence that disrupts expression of the gene(s) of interest. Cell lines with partially or completely inactivated genes are also provided.

In some embodiments, the endonucleases or subject site-directed modifying polypeptide can be guided by a RNA moledule. The RNA molecule that binds to the site-directed modifying polypeptide and targets the polypeptide to a specific location within the target DNA is referred to herein as the “DNA-targeting RNA” or “DNA-targeting RNA polynucleotide” (also referred to herein as a “guide RNA” or “gRNA”). A subject DNA-targeting RNA comprises two segments, a “DNA-targeting segment” and a “protein-binding segment.” By “segment” it is meant a segment/section/region of a molecule, e.g., a contiguous stretch of nucleotides in an RNA. A segment can also mean a region/section of a complex such that a segment may comprise regions of more than one molecule. For example, in some cases the protein-binding segment (described below) of a DNA-targeting RNA is one RNA molecule and the protein-binding segment therefore comprises a region of that RNA molecule. In other cases, the protein-binding segment (described below) of a DNA-targeting RNA comprises two separate molecules that are hybridized along a region of complementarity. As an illustrative, non-limiting example, a protein-binding segment of a DNA-targeting RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and may include regions of RNA molecules that are of any total length and may or may not include regions with complementarity to other molecules.

Furthermore, the methods of targeted integration as described herein can also be used to integrate one or more exogenous sequences. The exogenous nucleic acid sequence can comprise, for example, one or more genes or cDNA molecules, or any type of coding or non-coding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, also, U.S. Patent Publication Nos. 2005/0064474, 20070218528, 2008/0131962 and 2011/0201055, incorporated herein by reference in their entireties.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylates, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP or TALE DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP or TALEN as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., T-cells).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP or TALE or CRISPR DNA-binding domain is fused to an activation domain, the ZFP or TALE or CRISPR DNA-binding domain and the activation domain are in operative linkage if, in the fusion polypeptide, the ZFP or TALE or CRISPR DNA-binding domain portion is able to bind its target site and/or its binding site, while the activation domain is able to up-regulate gene expression. When a fusion polypeptide in which a ZFP or TALE or CRISPR DNA-binding domain is fused to a cleavage domain, the ZFP or TALE or CRISPR DNA-binding domain and the cleavage domain are in operative linkage if, in the fusion polypeptide, the ZFP or TALE or CRISPR DNA-binding domain portion is able to bind its target site and/or its binding site, while the cleavage domain is able to cleave DNA in the vicinity of the target site.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one ore more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest.

The phrase “oxidative stress conditions” as used herein, refers to conditions that results in oxidative stress, resulting in e.g. destruction of cells and cellular components (e.g. mitochondria), causing cells to lose their structure and/or function, and/or cell death. Particular oxidative stress conditions are those that result in or are related with mitochondrial dysfunction.

As used herein the phrase “oxidative stress” refers to an undesirable imbalance where in general oxidants outnumber antioxidants. This situation can particularly arise if the rate of ROS production overwhelms existing antioxidant defenses. In such circumstances, a series of cellular responses (e.g. mitochondrial dysfunction and the subsequent impaired respiratory chain and cellular respiration) can occur that can lead to an even greater increase in ROS production. Excessive ROS production and its otherwise ineffective regulation can be detrimental to cells and tissues, inducing cellular damage that ultimately can lead to cell death (apoptosis). Oxidative stress-associated damage also can cause undesirable changes to the structural and functional integrities of cells that can lead to the propagation of cells instead of apoptosis. Additionally, oxidatively-damaged cellular macromolecules can trigger immune responses that can lead to disease. See generally, D. G. Lindsay et al. (2002) Mol. Aspects of Med. 23: 1-38. In the case of plants, oxidative stress occurs e.g. in situations of ozone stress, in cases of necrosis as a result of pathogen infection or wounding, in cases of senescence and due to application of certain herbicides (like atrazine or paraquat).

Nucleases

Described herein are compositions, particularly nucleases, which are useful in correction of a mutant LRRK2 allele and/or mutation of an LRRK2 allele, for example to generate models of PD and/or cancer. The correction of a mutant LRRK2 allele and/or mutation of an LRRK2 allele can be achieved using any genome editing methods known in the art. Non-limiting examples include zinc finger nucleases (ZFNs), TAL-effector nucleases (TALENs) and Clustered Regularly Interspaced Short Palindromic Repeats (CRISPR) system.

In certain embodiments, the nuclease or subject site-directed modifying polypeptide is naturally occurring. In other embodiments, the nuclease or subject site-directed modifying polypeptide is non-naturally occurring, i.e., engineered in the DNA-binding domain and/or cleavage domain. For example, the DNA-binding domain of a naturally-occurring nuclease or subject site-directed modifying polypeptide may be altered to bind to a selected target site (e.g., a meganuclease that has been engineered to bind to site different than the cognate binding site). In other embodiments, the nuclease comprises heterologous DNA-binding and cleavage domains (e.g., zinc finger nucleases (ZFNs); TAL-effector nucleases (TALENs); meganuclease DNA-binding domains with heterologous cleavage domains, CRISPR/Cas nuclease protein).

In some cases, the nuclease can comprise an amino acid sequence having at most about 20%, at most about 30%, at most about 40%, at most about 50%, at most about 60%, at most about 70%, at most about 75%, at most about 80%, at most about 85%, at most about 90%, at most about 95%, at most about 99%, or 100%, amino acid sequence identity and/or homology to a wild type reference nuclease. The nuclease can comprise an amino acid sequence having at least about 20%, at least about 30%, at least about 40%, at least about 50%, at least about 60%, at least about 70%, at least about 75%, at least about 80%, at least about 85%, at least about 90%, at least about 95%, at least about 99%, or 100%, amino acid sequence identity and/or homology to a wild type reference nuclease. In some embodiments, the reference nuclease is a zinc finger nuclease. In some embodiments, the reference nuclease is a TAL-effector nuclease. In some instances, the reference nuclease can be a Cas6 family member (e.g., Csy4, Cas6). In some instances, the reference nuclease can be a Cas5 family member (e.g., Cas5 from D. vulgaris). In some instances, the reference nuclease can be a Type I CRISPR family member (e.g., Cas3). In some instances, the reference nucleases can be a Type II family member. In some instances, the reference nuclease can be a Type III family member (e.g., Cas6). A reference nuclease can be a member of the Repeat Associated Mysterious Protein (RAMP) superfamily (e.g., Cas7).

The nuclease can comprise amino acid modifications (e.g., substitutions, deletions, additions etc). In some instances, the nuclease can comprise one or more non-native sequences (e.g., a fusion, an affinity tag). The amino acid modifications may not substantially alter the activity of the nuclease. An nuclease comprising amino acid modifications and/or fusions can retain at least about 75%, at least about 80%, at least about 85%, at least about 90%, at least about 95%, at least about 97% or 100% activity of the wild-type nuclease.

In some instances, the modification can result alteration of the enzymatic activity of the nuclease. The modification can result in less than 90%, less than 80%, less than 70%, less than 60%, less than 50%, less than 40%, less than 30%, less than 20%, less than 10%, less than 5%, or less than 1% of the nuclease. In some instances, the modification occurs in the nuclease domain of an nuclease. Such modifications can result in less than 90%, less than 80%, less than 70%, less than 60%, less than 50%, less than 40%, less than 30%, less than 20%, less than 10%, less than 5%, or less than 1% of the nucleic acid-cleaving ability in one or more of the plurality of nucleic acid-cleaving domains of the wild-type nuclease.

In some embodiments, the DNA binding and targeting domain can be a RNA. The subject DNA-targeting RNA and a subject site-directed modifying polypeptide can form a complex. The DNA-targeting RNA provides target specificity to the complex by comprising a nucleotide sequence that is complementary to a sequence of a target DNA. The site-directed modifying polypeptide of the complex provides the site-specific activity. In other words, the site-directed modifying polypeptide is guided to a DNA sequence (e.g. a chromosomal sequence or an extrachromosomal sequence, e.g. an episomal sequence, a minicircle sequence, a mitochondrial sequence, a chloroplast sequence, etc.) by virtue of its association with at least the protein-binding segment of the DNA-targeting RNA (described above).

Exemplary naturally-occurring site-directed modifying polypeptides for CRISPR/Cas system are set forth in SEQ ID NOs:1-255 as a non-limiting and non-exhaustive list of naturally occurring Cas9/Csnl endonucleases. These naturally occurring polypeptides, as disclosed herein, bind a DNA-targeting RNA, are thereby directed to a specific sequence within a target DNA, and cleave the target DNA to generate a double strand break. A subject site-directed modifying polypeptide comprises two portions, an RNA-binding portion and an activity portion. In some embodiments, a subject site-directed modifying polypeptide comprises: (i) an RNA-binding portion that interacts with a DNA-targeting RNA, wherein the DNA-targeting RNA comprises a nucleotide sequence that is complementary to a sequence in a target DNA; and (ii) an activity portion that exhibits site-directed enzymatic activity (e.g., activity for DNA methylation, activity for DNA cleavage, activity for histone acetylation, activity for histone methylation, etc.), wherein the site of enzymatic activity is determined by the DNA-targeting RNA.

In some cases, a subject site-directed modifying polypeptide has enzymatic activity that modifies target DNA (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity).

In other cases, a subject site-directed modifying polypeptide has enzymatic activity that modifies a polypeptide (e.g., a histone) associated with target DNA (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

A. Nucleic Acid-Binding Domains

The genomic regions in a cell can be modified by any known systems in the art. The genome regions in a cell can be modified or edited in a site-specific fashion with a targeted DNA sequence. The systems for genome editing can target and bind to a domain of DNA sequences in the cell.

The nucleic acid or DNA-binding domain can comprise a region that contacts a nucleic acid. A nucleic acid-binding domain can comprise a nucleic acid. A nucleic acid-binding domain can comprise a proteinaceous material. A nucleic acid-binding domain can comprise nucleic acid and a proteinaceous material. A nucleic acid-binding domain can comprise RNA. There can be a single nucleic acid-binding domain. Examples of nucleic acid-binding domains can include, but are not limited to, a helix-turn-helix domain, a zinc finger domain, a leucine zipper (bZIP) domain, a winged helix domain, a winged helix turn helix domain, a helix-loop-helix domain, a HMG-box domain, a Wor3 domain, an immunoglobulin domain, a B3 domain, a TALE domain, a RNA-recognition motif domain, a double-stranded RNA-binding motif domain, a double-stranded nucleic acid binding domain, a single-stranded nucleic acid binding domains, a KH domain, a PUF domain, a RGG box domain, a DEAD/DEAH box domain, a PAZ domain, a Piwi domain, and a cold-shock domain.

In some instances, two or more nucleic acid-binding domains can be linked together. Linking a plurality of nucleic acid-binding domains together can provide increased polynucleotide targeting specificity. Two or more nucleic acid-binding domains can be linked via one or more linkers. The linker can be a flexible linker. Linkers can comprise 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length. Linkers can comprise at least 5%, 10%, 15%, 20%, 25%, 30%, 35%, 40%, 45%, 50%, 55%, 60%, 65%, 70%, 75%, 80%, 85%, 90%, 95%, or 100% glycine content. Linkers can comprise at most 5%, 10%, 15%, 20% 25%, 30%, 35%, 40%, 45%, 50%, 55%, 60%, 65%, 70%, 75%, 80%, 85%, 90%, 95%, or 100% glycine content. Linkers can comprise at least 5%, 10%, 15%, 20%, 25%, 30%, 35%, 40%, 45%, 50%, 55%, 60%, 65%, 70%, 75%, 80%, 85%, 90%, 95′%©, or 100% serine content. Linkers can comprise at most 5%, 10%, 15%, 20%, 25%, 30%, 35%, 40%, 45%, 50%, 55%, 60%, 65%, 70%, 75%, 80%, 85%, 90%, 95%, or 100% serine content.

Nucleic acid-binding domains may function to bind to nucleic acid sequences. In some instances, nucleic acid binding domains can bind to nucleic acids through hybridization. Nucleic acid-binding domains can be engineered (e.g. engineered to hybridize to a sequence in a genome). A nucleic acid-binding domain can be engineered by molecular cloning techniques (e.g., directed evolution, site-specific mutation, and rational mutagenesis).

In certain embodiments, the nuclease is a meganuclease (homing endonuclease). Naturally-occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

In certain embodiments, the nuclease comprises an engineered (non-naturally occurring) homing endonuclease (meganuclease). The recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 20070117128. The DNA-binding domains of the homing endonucleases and meganucleases may be altered in the context of the nuclease as a whole (i.e., such that the nuclease includes the cognate cleavage domain) or may be fused to a heterologous cleavage domain.

In other embodiments, the DNA-binding domain comprises a naturally occurring or engineered (non-naturally occurring) TAL effector DNA binding domain. See, e.g., U.S. Patent Publication No. 20110301073, incorporated by reference in its entirety herein. The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like effectors (TALE) which mimic plant transcriptional activators and manipulate the plant transcriptome (see Kay et at (2007) Science 318:648-651). These proteins contain a DNA binding domain and a transcriptional activation domain. One of the most well characterized TALEs is AvrBs3 from Xanthomonas campestgris pv. Vesicatoria (see Bonas et at (1989) Mol Gen Genet 218: 127-136 and WO2010079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et at (2006) J Plant Physiol 163(3): 256-272). In addition, in the phytopathogenic bacteria Ralstonia solanacearum two genes, designated brgl1 and hpx17 have been found that are homologous to the AvrBs3 family of Xanthomonas in the R. solanacearum biovar 1 strain GMI1000 and in the biovar 4 strain RS1000 (See Heuer et at (2007) Appl and Envir Micro 73(13): 4379-4384). These genes are 98.9% identical in nucleotide sequence to each other but differ by a deletion of 1,575 by in the repeat domain of hpx17. However, both gene products have less than 40% sequence identity with AvrBs3 family proteins of Xanthomonas.

Specificity of these TALEs depends on the sequences found in the tandem repeats. The repeated sequence comprises approximately 102 bp and the repeats are typically 91-100% homologous with each other (Bonas et al, ibid). Polymorphism of the repeats is usually located at positions 12 and 13 and there appears to be a one-to-one correspondence between the identity of the hypervariable diresidues at positions 12 and 13 with the identity of the contiguous nucleotides in the TALE's target sequence (see Moscou and Bogdanove, (2009) Science 326:1501 and Boch et al (2009) Science 326:1509-1512). Experimentally, the code for DNA recognition of these TALEs has been determined such that an HD sequence at positions 12 and 13 leads to a binding to cytosine (C), NG binds to T, NI to A, C, G or T, NN binds to A or G, and IG binds to T. These DNA binding repeats have been assembled into proteins with new combinations and numbers of repeats, to make artificial transcription factors that are able to interact with new sequences and activate the expression of a non-endogenous reporter gene in plant cells (Boch et al, ibid). Engineered TAL proteins have been linked to a FokI cleavage half domain to yield a TAL effector domain nuclease fusion (TALEN) exhibiting activity in a yeast reporter assay (plasmid based target). Christian et at ((2010)<Genetics epub 10.1534/genetics.110.120717). See, also, U.S. Patent Publication No. 20110301073, incorporated by reference in its entirety.

In certain embodiments, the DNA binding domain comprises a zinc finger protein. Preferably, the zinc finger protein is non-naturally occurring in that it is engineered to bind to a target site of choice. See, for example, See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties. Exemplary wild-type and mutant LRRK2 binding zinc finger proteins are described in U.S. Patent Publication No. 20120214241, incorporated by reference in its entirety herein.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in co-owned WO 02/077227.

In addition, as disclosed in these and other references, DNA domains (e.g., multi-fingered zinc finger proteins) may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The zinc finger proteins described herein may include any combination of suitable linkers between the individual zinc fingers of the protein. In addition, enhancement of binding specificity for zinc finger binding domains has been described, for example, in co-owned WO 02/077227.

Selection of target sites; ZFPs and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536 and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

CRISPR can be a genomic locus found in the genomes of many prokaryotes (e.g., bacteria and archaea), and can provide resistance to foreign invaders such as virus and phages, functioning as a type of immune system to help defend prokaryotes against foreign invaders. There can be three stages of CRISPR locus function: integration of new sequences into the locus, biogenesis of CRISPR RNA (crRNA), and silencing of foreign invader nucleic acid. There can be four types of CRISPR systems (e.g., Type I, Type II, Type III, TypeU).

A CRISPR locus can include a number of short repeating sequences referred to as “repeats.” The repeats can diverge between species and occur in clusters. Repeats can form hairpin structures and/or repeats can be unstructured single-stranded sequences. A CRISPR locus can comprise polynucleotide sequences encoding for Crispr Associated Genes (Cas) genes. Cas genes can display extreme sequence (e.g., primary sequence) divergence between species and homologues. For example, Casl homologues can comprise less than 15% primary sequence identity between homologues. Some Cas genes can comprise homologous secondary and/or tertiary structures. For example, despite extreme sequence divergence, many members of the Cas6 family of CRISPR proteins comprise a N-terminal ferredoxin-like fold. Cas genes can be named according to the organism from which they are derived. For example, Cas genes in Staphylococcus epidermidis can be referred to as Csm-type, Cas genes in Streptococcus thermophilus can be referred to as Csn-type, and Cas genes in Pyrococcus furiosus can be referred to as Cmr-type. Repeats can be regularly interspaced with unique intervening sequences referred to as “spacers,” resulting in a repeat-spacer-repeat locus architecture. Spacers can be identical to or have high homology with known foreign invader sequences. A spacer-repeat unit can encode a crisprRNA (crRNA). A crRNA can refer to the mature form of the spacer-repeat unit. A crRNA can comprise a “seed” sequence that can be involved in targeting a target nucleic acid (e.g., possibly as a surveillance mechansim against foreign nucleic acid). A seed sequence can be located at the 5′ or 3′ end of the crRNA. Various exemplified methods for the use of CRISPR systems and the modification of the system can be found in Deltcheva et al. CRISPR RNA maturation by trans-encoded small RNA and host factor RNase III. Nature 471(7340):602-7 (2011); M. M. Jinek, et al. A programmable dual-RNA-guided DNA endonuclease in adaptive bacterial immunity. Science 337, 816-821 (2012).; A. Marraffini, E. J. Sontheimer, Self versus non-self discrimination during CRISPR RNA-directed immunity. Nature 463, 568 (2010); Wang et al. One-step generation of mice carrying mutations in multiple genes by CRISPR/Cas-mediated genome engineering. Cell 153(4):910-8. (2013); Cong, L., et al., Multiplex genome engineering using CRISPR/Cas systems. Science 2013; 339(6121):819-23; Mali, P., et al., RNA-guided human genome engineering via Cas9. Science 2013; 339(6121):823-6.

B. Cleavage Domains

The nucleic acid-cleaving domain can be a nucleic acid-cleaving domain from any nucleic acid-cleaving protein. The nucleic acid-cleaving domain can originate from a nuclease. Suitable nucleic acid-cleaving domains include the nucleic acid-cleaving domain of endonucleases (e.g., AP endonuclease, RecBCD enonuclease, T7 endonuclease, T4 endonuclease IV, Bal 31 endonuclease, EndonucleaseI (endo I), Micrococcal nuclease, Endonuclease II (endo VI, exo III)), exonucleases, restriction nucleases, endoribonucleases, exoribonucleases, RNases (e.g., RNAse I, II, or III). In some instances the nucleic acid-cleaving domain can originate from the FokI endonuclease. A site-directed polypeptide can comprise a plurality of nucleic acid-cleaving domains. Nucleic acid-cleaving domains can be linked together. Two or more nucleic acid-cleaving domains can be linked via a linker. In some embodiments, the linker can be a flexible linker. Linkers can comprise 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length. In some embodiments, a site-directed polypeptide can comprise the plurality of nucleic acid-cleaving domains.

In some embodiments, the genome editing system can be the CRISPR system. The CRISPR system can contain a site-directed polypeptide (e.g., Cas9 in a CRISPR system) that can comprise a plurality of nuclease domains. Cas9 can comprise a HNH or HNH-like nuclease domain and/or a RuvC or RuvC-like nuclease domain. HNH or HNH-like domains can comprise a McrA-like fold. HNH or FINE-like domains can comprise two antiparallel β-strands and an α-helix. HNH or FINE-like domains can comprise a metal binding site (e.g., divalent cation binding site). HNH or HNH-like domains can cleave one strand of a target nucleic acid (e.g., complementary strand of the crRNA targeted strand). Proteins that comprise an HNH or HNH-like domain can include endonucleases, clicins, restriction endonucleases, transposases, and DNA packaging factors.

RuvC or RuvC-like domains in a site-specific genome editing system (e.g., CRISPR) can comprise anRNaseH or RNaseH-like fold. RuvC/RNaseH domains can be involved in a diverse set of nucleic acid-based functions including acting on both RNA and DNA. The RNaseH domain can comprise 5 β-strands surrounded by a plurality of α-helices. RuvC/RNaseH or RuvC/RNaseH-like domains can comprise a metal binding site (e.g., divalent cation binding site). RuvC/RNaseH or RuvC/RNaseH-like domains can cleave one strand of a target nucleic acid (e.g., non-complementary strand of the crRNA targeted strand). Proteins that comprise a RuvC, RuvC-like, or RNaseH-like domain can include RNaseH, RuvC, DNA transposases, retroviral integrases, and Argonaut proteins).

In some instances, a site-directed polypeptide can comprise a highly basic patch. A highly basic patch can recognize a PAM motif. A RuvC and/or a RuvC-like domain can comprise a highly basic patch.

Any suitable cleavage domain can be operatively linked to a DNA-binding domain to form a nuclease. For example, ZFP DNA-binding domains have been fused to nuclease domains to create ZFNs—a functional entity that is able to recognize its intended nucleic acid target through its engineered (ZFP) DNA binding domain and cause the DNA to be cut near the ZFP binding site via the nuclease activity. See, e.g., Kim et al. (1996) Proc Nat'l Acad Sci USA 93(3):1156-1160. More recently, ZFNs have been used for genome modification in a variety of organisms. See, for example, United States Patent Publications 20030232410; 20050208489; 20050026157; 20050064474; 20060188987; 20060063231; and International Publication WO 07/014275.

As noted above, the cleavage domain may be heterologous to the DNA-binding domain, for example a zinc finger DNA-binding domain and a cleavage domain from a nuclease or a TALEN DNA-binding domain and a cleavage domain, or a CRISPR/Cas nucleic acid binding domain and cleavage domain from a nuclease, or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However any integral number of nucleotides or nucleotide pairs can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a DNA binding domain and two Fok I cleavage half-domains can also be used. Exemplary Type IIS restriction enzymes are described in International Publication WO 07/014275, incorporated herein in its entirety. Additional restriction enzymes also contain separable binding and cleavage domains, and these are contemplated by the present disclosure. See, for example, Roberts et al. (2003) Nucleic Acids Res. 31:418-420.

A cleavage domain or cleavage half-domain can be any portion of a protein that retains cleavage activity, or that retains the ability to multimerize (e.g., dimerize) to form a functional cleavage domain.

In certain embodiments, the cleavage domain comprises one or more engineered cleavage half-domain (also referred to as dimerization domain mutants) that minimize or prevent homodimerization, as described, for example, in U.S. Patent Publication Nos. 20050064474; 20060188987; 20080131962 and 20110201055, the disclosures of all of which are incorporated by reference in their entireties herein. Amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains.

Exemplary engineered cleavage half-domains of Fok I that form obligate heterodimers include a pair in which a first cleavage half-domain includes mutations at amino acid residues at positions 490 and 538 of Fok I and a second cleavage half-domain includes mutations at amino acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. See, e.g., U.S. Patent Publication Nos. 2008/0131962, the disclosure of which is incorporated by reference in its entirety for all purposes. In certain embodiments, the engineered cleavage half-domain comprises mutations at positions 486, 499 and 496 (numbered relative to wild-type FokI), for instance mutations that replace the wild type Gln (Q) residue at position 486 with a Glu (E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In other embodiments, the engineered cleavage half-domain comprises mutations at positions 490, 538 and 537 (numbered relative to wild-type FokI), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In other embodiments, the engineered cleavage half-domain comprises mutations at positions 490 and 537 (numbered relative to wild-type FokI), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). (See US Publication No. 2011/0201055). Engineered cleavage half-domains described herein can be prepared using any suitable method, for example, by site-directed mutagenesis of wild-type cleavage half-domains (Fok I) as described in U.S. Patent Publication Nos. 20050064474 and 20080131962.

Alternatively, nucleases may be assembled in vivo at the nucleic acid target site using so-called “split-enzyme” technology (see e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in WO 2009/042163 and 20090068164. Nuclease expression constructs can be readily designed using methods known in the art. See, e.g., United States Patent Publications 20030232410; 20050208489; 20050026157; 20050064474; 20060188987; 20060063231; and International Publication WO 07/014275. Expression of the nuclease may be under the control of a constitutive promoter or an inducible promoter, for example the galactokinase promoter which is activated (de-repressed) in the presence of raffinose and/or galactose and repressed in presence of glucose.

### Target Sites

As described in detail above, DNA domains can be engineered to bind to any sequence of choice in an LRRK2 locus. An engineered DNA-binding domain can have a novel binding specificity, compared to a naturally-occurring DNA-binding domain. Engineering methods include, but are not limited to, rational design and various types of selection. Rational design includes, for example, using databases comprising triplet (or quadruplet) nucleotide sequences and individual (e.g., zinc finger) amino acid sequences, in which each triplet or quadruplet nucleotide sequence is associated with one or more amino acid sequences of DNA binding domain which bind the particular triplet or quadruplet sequence. See, for example, co-owned U.S. Pat. Nos. 6,453,242 and 6,534,261, incorporated by reference herein in their entireties. Rational design of TAL-effector domains can also be performed. See, e.g., U.S. Patent Publication No. 20110301073. Rational design of CRISPR target domain can be performed. See U.S. Patent Publication No. 20100093617 A1.

Exemplary selection methods applicable to DNA-binding domains, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237.

Selection of target sites; nucleases and methods for design and construction of fusion proteins (and polynucleotides encoding same) are known to those of skill in the art and described in detail in U.S. Patent Application Publication Nos. 20050064474 and 20060188987, incorporated by reference in their entireties herein.

In addition, as disclosed in these and other references, DNA-binding domains (e.g., multi-fingered zinc finger proteins) may be linked together using any suitable linker sequences, including for example, linkers of 5 or more amino acids. See, e.g., U.S. Pat. Nos. 6,479,626; 6,903,185; and 7,153,949 for exemplary linker sequences 6 or more amino acids in length. The proteins described herein may include any combination of suitable linkers between the individual DNA-binding domains of the protein. See, also, U.S. Patent Publication No. 20110287512.

### Donors

As noted above, alteration of an LRRK2 gene can include insertion of an exogenous sequence (also called a “donor sequence” or “donor”). It will be readily apparent that the donor sequence is typically not identical to the genomic sequence that it replaces. For example, the sequence of the donor polynucleotide can contain one or more single base changes, insertions, deletions, inversions or rearrangements with respect to the genomic sequence, so long as sufficient homology with chromosomal sequences is present. Alternatively, a donor sequence can contain a non-homologous sequence flanked by two regions of homology. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded or double-stranded and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the LRRK2 gene. However, it will be apparent that the donor may comprise a promoter and/or enhancer, for example a constitutive promoter or an inducible or tissue specific promoter.

Furthermore, although not required for expression, exogenous sequences may also be transcriptional or translational regulatory sequences, for example, promoters, enhancers, insulators, internal ribosome entry sites, sequences encoding 2A peptides and/or polyadenylation signals.

### Delivery

The nucleases, polynucleotides encoding these nucleases, donor polynucleotides and compositions comprising the proteins and/or polynucleotides described herein may be delivered in vivo or ex vivo by any suitable means.

Methods of delivering nucleases as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Nucleases and/or donor constructs as described herein may also be delivered using vectors containing sequences encoding one or more of the zinc finger, TALEN protein(s) or CRISPR/Cas proteins. Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more of the sequences needed for treatment. Thus, when one or more nucleases and a donor construct are introduced into the cell, the nucleases and/or donor polynucleotide may be carried on the same vector or on different vectors. When multiple vectors are used, each vector may comprise a sequence encoding one or multiple nucleases and/or donor constructs.

Conventional viral and non-viral based gene transfer methods can be used to introduce nucleic acids encoding nucleases and donor constructs in cells (e.g., mammalian cells) and target tissues. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds.) (1995); and Yu et al., Gene Therapy 1:13-26 (1994).

Methods of non-viral delivery of nucleic acids include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424, WO 91/16024.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et at (2009) Nature Biotechnology 27(7):643).

The use of RNA or DNA viral based systems for the delivery of nucleic acids encoding engineered ZFPs take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of ZFPs include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications in which transient expression is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoff et al., Hum. Gene Ther. 1:111-2 (1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). Other AAV serotypes, including AAV1, AAV3, AAV4, AAV5, AAV6 and AAV8, can also be used in accordance with the present invention.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity. An example of the use of an Ad vector in a clinical trial involved polynucleotide therapy for anti-tumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Vectors (e.g., retroviruses, adenoviruses, liposomes, etc.) containing nucleases and/or donor constructs can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of polynucleotides described herein include non-integrating lentivirus vectors (IDLV). See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222; U.S. Patent Publication No 2009/054985.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

It will be apparent that the nuclease-encoding sequences and donor constructs can be delivered using the same or different systems. For example, a donor polynucleotide can be carried by a plasmid, while the one or more nucleases can be carried by a AAV vector. Furthermore, the different vectors can be administered by the same or different routes (intramuscular injection, tail vein injection, other intravenous injection, intraperitoneal administration and/or intramuscular injection. The vectors can be delivered simultaneously or in any sequential order.

Formulations for both ex vivo and in vivo administrations include suspensions in liquid or emulsified liquids. The active ingredients often are mixed with excipients which are pharmaceutically acceptable and compatible with the active ingredient. Suitable excipients include, for example, water, saline, dextrose, glycerol, ethanol or the like, and combinations thereof. In addition, the composition may contain minor amounts of auxiliary substances, such as, wetting or emulsifying agents, pH buffering agents, stabilizing agents or other reagents that enhance the effectiveness of the pharmaceutical composition.

### Parkinson's Disease, Cancer and LRRK2

As described above, mutations in LRRK2 are found in both familial and sporadic cases of PD. The instant invention describes methods and compositions that can be used to introduce or repair such mutations.

In particular, specific mutations encoded by mutant LRRK2 genes that have been proven shown to be pathogenic in the development of PD include Y1699C, R1441C, R1441H, R1441H, I1371V, Y1699G, G2019S, 12020T, and G2385R. Mutations within LRRK2 that are potentially pathogenic include E334K, Q1111H, I1192V, I1122V, 51228T, A1442P, L1719F, and T2356I. Those mutations that are associated with an increased risk of developing PD are R1628P and G2385R (see Kumari, ibicl). Thus, the methods and compositions of the instant invention are useful for repairing such mutations in LRRK2, and are useful for developing cell and transgenic animal models to study the intracellular pathology associated with LRRK2 mutations and for studying the whole organism consequences of these mutations.

Mutations in LRRK2 may also be implicated in some types of cancers, including but not limited to, melanomas, renal and thyroid cancers. Thus, tools designed to knock out or knock in specific LRRK2 mutations such as the G2019S LRRK2 mutation in cancer models will be useful in furthering an understanding of the underlying biology and in the development of specific drug therapies. Further, specific nucleases targeted to a specific LRRK2 mutation can be employed to knock out or correct the mutation. Nucleases can also be used to cause the insertion of an LRRK2 mutation-specific tag in order to develop cell lines for the investigation of LRRK2 mutation specific therapeutics.

The cells, cell lines and transgenic animals, for example, isogenic cell lines, as described herein are useful for drug development. Such cells and animals may reveal phenotypes associated with a particular mutation (e.g. LRRK2 G2019S alterations) or with its correction, and may be used to screen drugs that will interact either specifically with the mutation(s) or mutant proteins in question, or that are useful for treatment of the disease in an afflicted animal. Therapeutically, iPSCs can be derived ex vivo from a patient afflicted with a known genetic mutation associated with PD, and this mutation can be corrected using ZFN- or TALEN-mediated gene correction. The corrected iPSC can then be differentiated into dopaminergic neurons and reimplanted into the patient. Alternatively, the ZFNs may be introduced into the patient's cells in vivo for in situ gene correction. These cell lines can also provide tools to investigate the effects of specific mutations patient-specific iPS cell lines that are only different by the disease-causing mutation, thus representing a ‘genetically virtually identical’ control cell line. The resulting isogenic panel of iPSCs that carry different allelic forms of LRRK2 at the endogenous locus provides a genetic tool for repair of disease-specific mutations, drug screening and discovery, and disease mechanism research.

The availability of patient-specific iPS cell lines with both repaired and induced mutations and their isogenic controls are also useful in a wide-variety of medical applications, including but not limited to, the study of mechanisms by which these mutations cause disease to translating these “laboratory cures” to treatments for patients who actually manifest disease induced by these mutations.

The following Examples relate to exemplary embodiments of the present disclosure in which the nuclease comprises a zinc finger nuclease (ZFN). It will be appreciated that this is for purposes of exemplification only and that other nucleases can be used, for instance homing endonucleases (meganucleases) with engineered DNA-binding domains and/or fusions of naturally occurring of engineered homing endonucleases (meganucleases) DNA-binding domains and heterologous cleavage domains or TALENs.

### Oxidative Stress

Mutation of LRRK2 may be related to the sensitivity and response to oxidative challenges and stress of cells. For example without limitation, both the basal reactive oxygen species (ROS) production rates and their response to an inducer of chronic ROS stress (TBHP). Various methods of modulating oxidative stress can be found in WO 2013155166 A1, WO 2012075549 and WO 2003032968 A1. The cell lines or organisms with LRRK2 mutation may be used as models for screening compounds for modulating the oxidative stress.

Oxidative stress may play a critical role in the pathogenesis of several diseases including atherosclerosis, diabetes or other metabolic syndromes or Parkinson's disease. All these conditions are also accompanied by the presence of an oxidative stress, and oxidative stress may be a mechanism in the development of insulin resistance. there have been reports on many diseases such as diabetes, nervous diseases, renal diseases, hepatic cirrhosis, arthritis, retinopathy of prematurity, ocular uveitis, retinal rust disease, senile cataract, side-effect failures due to radiation therapy, asbestos diseases, bronchial failures due to smoking, anticancer drug side-effect failures, cerebral edema, pulmonary edema, foot edema, cerebral infarction, hemolytic anemia, progeria, spilepsy, Alzheimer disease, Down syndrome, Parkinson disease, Behect's disease, Crohn's disease, Kawasaki disease, Weber-Christian disease, collagen disease, progressive systemic sclerosis, herpetic dermatitis, immune deficiency syndrome, and the like. Although the active oxygen species causing oxidative stress may be originally necessary and essential for biological defense, excessive oxidative stress can be often present due to reductions of in-vivo antioxidative substances with changes in easting habits or increases in amount of lipids which easily produce release sources of free radicals. The oxidative stress may act as triggers or worsening factors of many diseases.

More in particular, as also described above, oxidative stress is implicated in mitochondrial dysfunction. Mitochondrial dysfunction has been established to contribute to the pathology of numerous diseases and is suspected in many more. In humans, many muscular and neurological disorders, various forms of cancer, diabetes, obesity, other disorders and ageing are associated with mitochondrial dysfunction (as discussed e.g. in Wallace, 2005, Annu Rev Genet 39, 359-407, Modica-Napolitano, 2004, Mitochondrion 4, 755-62 or Orth, 2001, Am J Med Genet 106, 27-36). A role for loss of mitochondrial function in normal aging has long been suspected. Most hypotheses focus on free radical damage to mitochondrial DNA. Mitochondrial dysfunction also plays a central role in the pathogenesis of several inborn errors of metabolism (e.g. Wilson's disease (WD) and inborn errors in respiratory chain complexes) but also in the frequent non-alcoholic fatty liver disease (NAFLD) or nonalcoholic steatohepatitis, which is the hepatic manifestation of the metabolic syndrome, and in other associated disorders as diabetes and obesity. In this respect, there is growing evidence that mitochondrial dysfunction, particularly respiratory chain deficiency, plays an role in the pathophysiology of NAFLD, which is linked to the generation of ROS by the damaged respiratory chain [0].

The responses and sensitivity to the oxidative stress may be measured by many different methods. For example without limitation, production of free radicals, mitochondrial membrane potential (MMP), mitochondrial transitional pore opening (MTP) and caspase activation. In some cases, the effect of lessening oxidative stress in vivo can be evaluated with urinary 8-hydroxydeoxyguanosine (80H-dG) having high effectiveness as an oxidative stress marker. This material can result from nucleic acid damage due to oxidative stress, and can be discharged into urine without undergoing further metabolism. It is thus thought that the amount of oxidative stress in vivo and the amount of 80H-dG discharged can have a close relationship therebetween. A decrease in amount of the material in urine can mean a reduction in oxidative stress in vivo and the prevention of damage to nucleic acid.

The present invention also provides methods for screening a compound for reducing the oxidative stress, comprising providing an isogenic cell line with a modified LRRK2; applying a condition of oxidative stress to the isogenic cell line; contacting the isogenic cell line under the condition of oxidative stress with a compound; and assaying the isogenic cell line for a response to the compound, thereby screening a compound for reducing sensitivity and/or response to oxidative stress. The compound can be used for treating an oxidative stress-related disorder, preferably a mitochondrial dysfunction related disorder, in a subject in need thereof, the method comprising administering to the subject a therapeutically effective amount of said compound, thereby treating or ameliorating the oxidative stress-related or mitochondrial dysfunction related disorder. Alternatively, the present invention also relates to the use of a compound of the present invention for the manufacture of a medicament for the prevention and/or treatment of oxidative stress related disorders and to the use of said compound for the screening of materials for their therapeutic activity.

Chemically, oxidative stress is associated with increased production of oxidizing species or a significant decrease in the effectiveness of antioxidant defenses, such as glutathione. The effects of oxidative stress depend upon the size of these changes, with a cell being able to overcome small perturbations and regain its original state. However, more severe oxidative stress can cause cell death and even moderate oxidation can trigger apoptosis, while more intense stresses may cause necrosis.

Production of reactive oxygen species is a particularly destructive aspect of oxidative* stress. Such species include free radicals and peroxides. Some of the less reactive of these species (such as superoxide) can be converted by oxidoreduction reactions with transition metals or other redox cycling compounds (including quinones) into more aggressive radical species that can cause extensive cellular damage. The major portion of long term effects is inflicted by damage on DNA. DNA damage can be induced by ionizing radiation is similar to oxidative stress, and these lesions have been implicated in aging and cancer. Biological effects of single-base damage by radiation or oxidation, such as 8-oxoguanine and thymine glycol, have been extensively studied. Recently has the focus shifted to some of the more complex lesions. Tandem DNA lesions are formed at substantial frequency by ionizing radiation and metal-catalyzed H2O2 reactions. Under anoxic conditions, the predo-minant double-base lesion is a species in which C8 of guanine is linked to the 5-methyl group of an adjacent 3′-thymine (G[8,5-Me]T). Most of these oxygen-derived species are produced at a low level by normal aerobic metabolism. Normal cellular defense mechanisms destroy most of these. Likewise, any damage to cells is constantly repaired. However, under the severe levels of oxidative stress that cause necrosis, the damage causes ATP depletion, preventing controlled apoptotic death and causing the cell to simply fall apart.

In some aspects, mitochondrial DNA damage is used to assess the severity or prognosis of Parkinson's disease of Parkinson's related disease, for example in patients carrying mutations in LRRK2 genes. By determining the level of mitochondrial DNA damage in a subject such as a patient diagnosed with Parkinson's disease or being at risk of Parkinson's disease, a person of skill in the art would be able to determine the relative onset of disease or the efficacy of therapy which the patient is undergoing. For example, the patient taking therapy for Parkinson's may evidence reversed or reduced mitochondrial DNA damage of a cell or organism. Alternatively, an increase in mitochondrial DNA damage would signal a worsening of the patient's condition. The isogenic cell lines or organisms with LRRK2 mutation may further be used as models for screening compounds for reducing or correcting mitochondrial DNA damage as discussed above. As used herein, the term “mtDNA damage” generally refers to any type of lesion (i.e. base alterations, apurinic sites, strand breaks, adduct formation, etc.) or mtDNA length mutation (deletions, insertions, and duplications) that can potentially be detected either directly by QPCR (by blocking the polymerase, or resulting in a QPCR product of size different than anticipated, i.e. □mtDNA length mutations), or in concert with an enzymatic action (i.e. DNA can be treated with FAPY glycosylase before QPCR to detect 8-oxo-G).” Any “downstream” or resultant effect damage of mitochondrial DNA will reflect the same disease process. For example, measurement of mitochondrial protein production, changes in mitochondria) oxidative phosphorylation or changes in mitochondrial ATP production would accomplish the same goal.

### EXAMPLES

### Example 1

### Generation of an Isogenic Panel of iPSCs Carrying Alleles of LRRK2

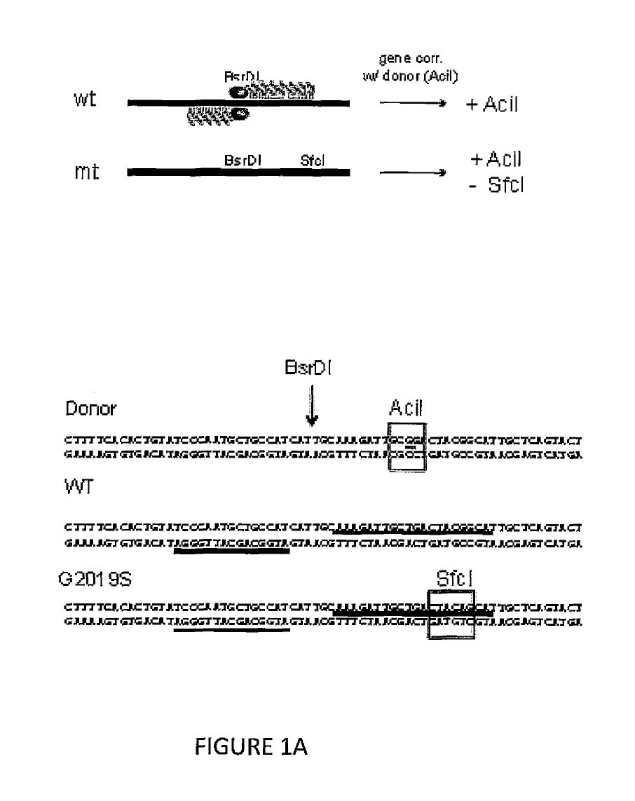

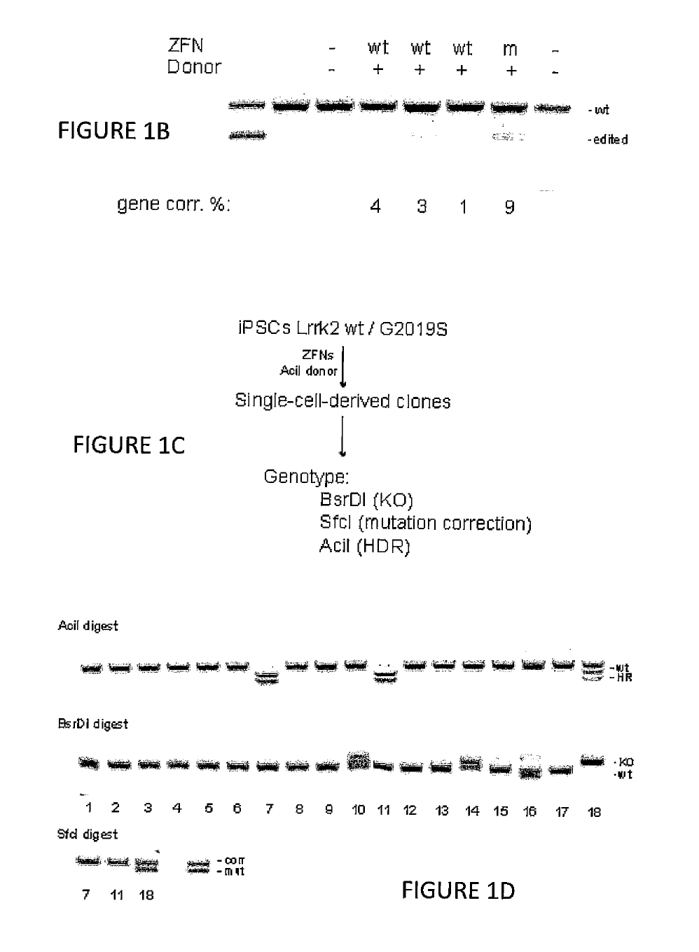

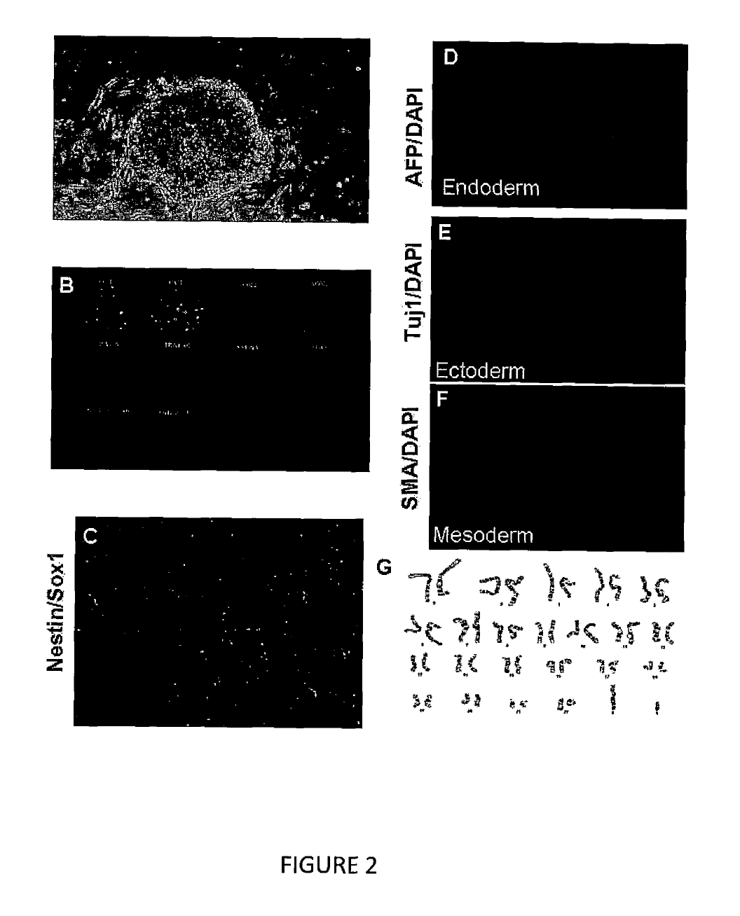

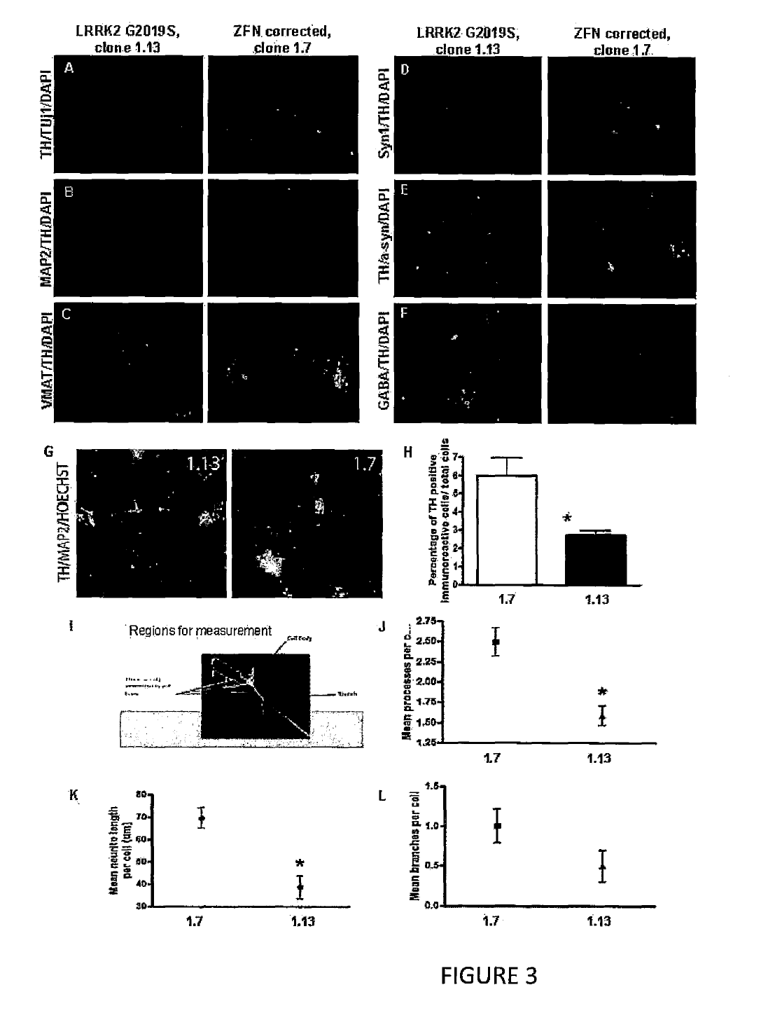

LRRK2 ZFNs that bind to wild-type or the G2019S mutant were prepared as described in U.S. Patent Application No. 20120214241. These ZFNs were used to correct the LRRK2 G2019S mutation from patient iPSCs as follows. A donor DNA construct that, with co-delivery of the ZFNs (typically as mRNA as described in Doyon et al. (2008) Nature Biotech. 26:702-708), would trigger homology-directed repair of the break and gene correction of the mutant allele (FIG. 1A).

Furthermore, we engineered a donor construct that would allow the rapid, accurate assessment of, first, the editing outcome itself (knockout vs. correction) and, second, the allele affected by the editing. We therefore introduced a silent AciI RFLP in the donor sequence. Co-delivery of this donor DNA with LRRK2 (wt-ZFN) or LRRK2 mutation-specifying ZFN (mut-ZFN) produced 9% targeted gene correction in patient-derived iPSCs heterozygous for the LRRK2 G2019S mutation (FIG. 1B).

All of the genome editing data shown so far have relied on assaying a pool of cells to measure genome editing frequency. Analysis of genome variant effect on function, however, requires a single-cell-derived clone with the desired novel allele. In the genome editing scheme (FIG. 1C) using this donor, each outcome is associated with a specific constellation of gain or a loss of particular RFLPs: (i) a knockout of either allele leads to the loss of a BsrDI site; (ii) repair of the ZFN-induced break using the donor as a template generates a novel AciI site; (iii) correction of the mutant allele eliminates an SfcI site.

LRRK2 wt/wt and G2019G patient-derived iPSCs were cultured in feeder-independent conditions using mTESR1 media (Stem Cell Technologies) and a BD Matrigel (Millipore) matrix following manufacturers' instructions. Cultures were maintained differentiation-free manually and expanded mechanically using the StemPro® EZPassage™ Disposable Stem Cell Passaging tool (Invitrogen).

We co-delivered the mutation-specific ZFNs and the corrective donor construct into iPSCs, performed limiting dilution in the absence of selection, and then performed a nested-PCR-based target locus genotyping of each clone (to insure against any contamination of the donor plasmid). An example of the clone derivation process and genotyping by restriction digest is shown in FIGS. 1C and D.

Remarkably, an unprecedented 78% of the clones carried a ZFN-driven allele at the endogenous locus. Only 16% of the single-cell derived clones remained unedited. As expected, given the prevalence of end joining over homology-directed repair pathways in human cells, 57% of the clones carried a small indel modification at the target locus, either in homozygous or heterozygous form. Of significant further note, 14% (14 out of 100 clones) carried an allele of LRRK2 that was corrected from its mutant to a wild-type form with the mutant ZFN. To exclude any possibility of a RFLP-based artifact, we sequenced all the PCR products used in this analysis; the sequencing data yielded frequencies congruent to those from the RFLP analysis.

Our data show that the process we have developed allows selection-free, sorting-free isolation of iPSCs carrying novel investigator-specified alleles at a frequency without precedent. Having established a single-cell-derived clones carrying novel engineered alleles of LRRK2, we next set out to determine whether the alteration of the cells' genomic status for this locus has any consequence for their cellular phenotype and whether the ZFN modified clones maintained their stemness, normal karyotype and potential for neuronal differentiation.

### Example 2

### Increased Sensitivity to Oxidative Stress in LRRK2 Parkinsonism is Ameliorated by ZFN Gene Correction in iPSC Derived Neural Cultures

We investigated if the corrected and mutant LRRK2, p.G2019S neural progenitor cells differ in their sensitivity and response to oxidative challenges and stress, both the basal reactive oxygen species (ROS) production rates and their response to an inducer of chronic ROS stress (TBHP) in live NPCs.

Cells were assayed for free radical production using CM-H₂DCFDA that can be oxidized and rendered fluorescent by hydroxyl radicals produced by mitochondria. See, e.g., Petronilli et al. (1999) Biophysical journal 76:725-734; Nicholls (2009) Biochimica et biophysica acta 1787:1416-1424. Cytoplasmic fluorescence of this indicator dye in viable cells was assayed by flow cytometry to obtain a signature for cellular oxidative stress.

In particular, for stress experiments, NPCs were seeded 24 hours before being cultured under the following selective media conditions for an additional 18 hours: Standard NPC growth medium as above (HG=high glucose), HG plus 20 μM rotenone (HG+R) (SIGMA # R-8875) or an NPC growth medium formulation based on HG medium without glucose (NG, Life Techn. Neurobasal-A, Formula 05-0128DJ).

For flow cytometry, NPCs were seeded at 80% confluence on either 24-well (1-2×10⁵ cells) or 48 well plates (5×10⁴ cells) (NUNC #s 142475, 150687) on Geltrex (100 ug/ml protein) coated plates 24 hours before the experiment and then challenged as described above. In general two replicate wells were analyzed in each experimental run. After the respective treatment, cells were sampled by flow cytometry with an Accuri™ C6 cytometer with C-Sampler™ (BD Accuri) and subsequently analyzed using the Cflow® analysis software.

For HCS microscopy, cells were seeded 24 hours before the experiment at 75% density on 96-well plates (1×10⁴) (Corning #3603 or Perkin Elmer ViewPlate-96F TC #6005182) that were thin-layer coated with Geltrex; (50 ug/ml).

High content screening by automated microscopy was with an ImageXpress Micro system). Image and data analysis was performed with the MetaXpress software modules for Granularity and Transfluor™ (Molecular Devices).

NPCs were seeded in 96-well plates and challenged with rotenone or no glucose as described above. Selected wells were also pretreated with the ROS inducer TBHP at 200 μM for 60 min. Cell pellets were then carefully resuspended in 100 μl HBSS Plus (HBSS, 10 mM HEPES, 2 mM L-Glutamine, 100 μM Na-Pyruvate) also containing a 1/20 dilution of an Alexa-Fluor 647 Annexin-V conjugated antibody (Life Techn. # A23204) and 10 μM 5-(and-6)-chloromethyl-2′,7′-dichloro-dihydro-fluorescein diacetate acetyl ester (CM-H₂DCFDA) (Life Techn. #C6827) and then transferred to a 96 well U-bottom microplate (Corning® #7007). Cells were then incubated for 30 min at room temperature in the dark with orbital agitation (100 rpm/min.) before assaying.

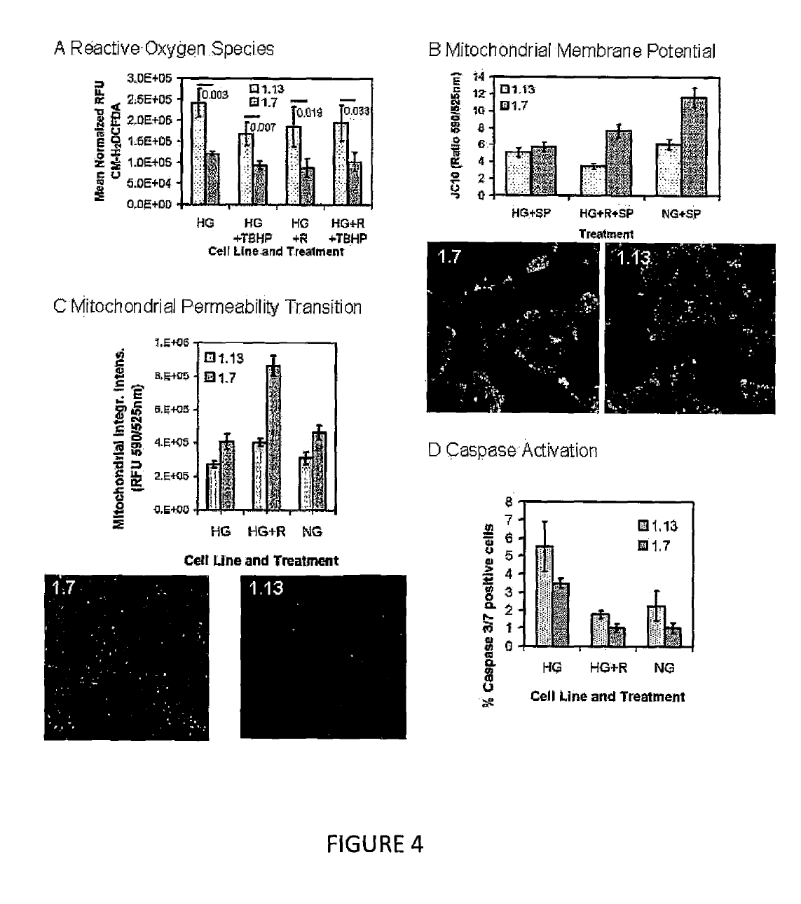

Analysis of nutritional or toxin challenged LRRK2 G2019S mutant viable cells loaded with fluorescent ROS indicator dye consistently showed higher oxidative stress levels both under normal growth conditions and with rotenone treatment compared to NPCs from the ZFN-corrected line (FIG. 4A). Both cell lines showed an increase of ROS in response to the radical generator TBHP when used at high levels under normal growth conditions, however, the ZFN-corrected cell line appeared to better mitigate the impact of functional mitochondrial impairment by rotenone under those conditions, possibly indicative of the improved ability of this cell line to cope with mitochondrial stress imposed by the environmental toxin rotenone.

### Example 3

### LRRK2 G2019S ZFN Editing in Neural Progenitors Cells Rescues Mitochondrial Membrane Potential

Mitochondrial membrane potential (MMP) in adherent cells was measured by the MMP-dependent fluorescent ratiometric dye JC-10. See, e.g., Polster & Fiskum, (2004) Journal of Neurochemistry 90:1281-1289; MacLeod et al. (2006) Neuron 52:587-593.

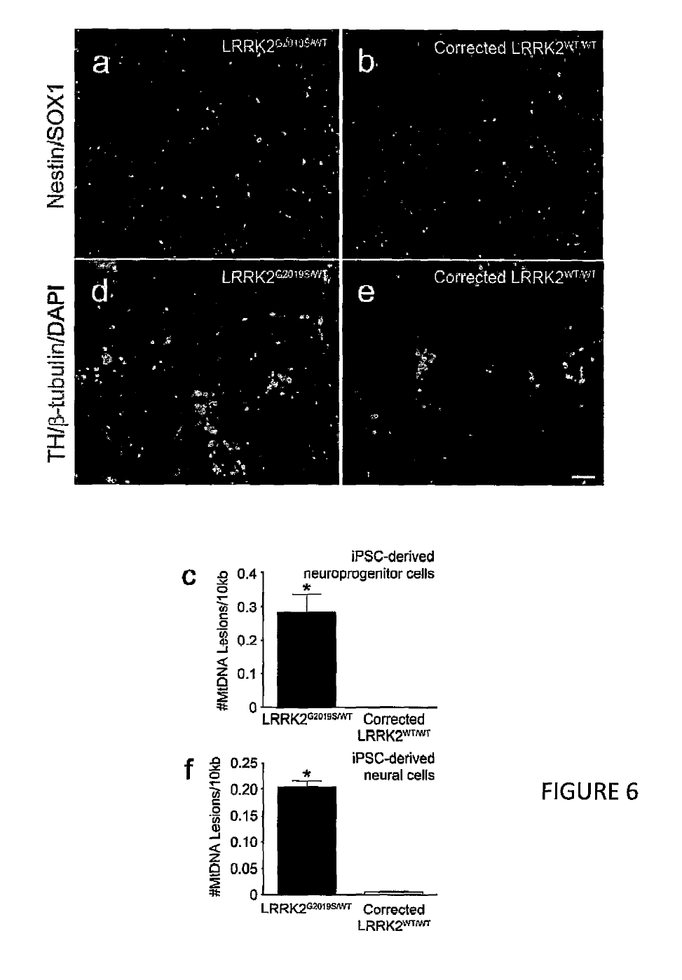

We detected significant changes in MMP in live cells. JC-10 is capable of selectively entering mitochondria where it reversibly forms J-aggregates if a significantly high MMP is present which results in fluorescence shift in emitted light from 520 nm (monomer) to 570 nm (J-aggregate). HCS microscopy and image analysis of mitochondrial JC-10 fluorescence patterns and dye ratios in cells challenged by the apoptosis inducer staurosporine revealed only minor differences in mitochondrial fluorescence staining patterns when comparing the LRRK2 G2019S mutant and ZFN-corrected line when assayed under normal growth conditions (FIG. 6B). When cells were stressed by rotenone and/or nutrient withdrawal, JC-10 aggregate-emitted red fluorescence, indicative of the presence of a high MMP and therefore mitochondrial membrane integrity and oxidative phosphorylation capacity, was more prominent in the ZFN-corrected cell line. This was observed both under no glucose alone and in presence of a pro-apoptotic toxic challenge with staurosporine.

### Example 4

### Mitochondrial Transition Pore Opening (MTP) is Impaired in LRRK2 Parkinsonism, but can be Genetically Rescued by ZFN Editing

Next, we investigated the integrity of the mitochondrial compartment in these cell lines by assaying the sensitivity of mitochondria to mitochondrial transition pore (MTP) opening, resulting in loss of mitochondrial membrane potential and induction of cellular apoptosis. Dachsel et al. (2010) Parkinsonism & related disorders 16: 650-655.

Mitochondrial MTP opening can be investigated by loading cells with the calcium chelator calcein. Upon cleavage of calcein AM by cellular esterases, free calcein can bind cellular calcium and emits strong fluorescence. We quenched cytoplasmic fluorescence by the mitochondrial impermeable CoCl₂ therefore had a selective signal of the mitochondria and examined retention of mitochondrial calcein signal and which describes mitochondrial membrane integrity under nutritional and toxicant stress. See, e.g., Ramonet et al. (2011) PloS one 6, e18568.

The LRRK2 mutant cell lines displayed accelerated rates of calcein loss under all experimental conditions compared to the ZFN corrected NPCs (FIG. 4C). Interestingly, corrected and LRRK2 mutant lines showed increased mitochondrial calcein loading under rotenone stress, indicative of the response of mitochondria to stress by the mitochondrial complex I inhibitor rotenone, resulting in hyperpolarization and increased calcein uptake (Winner et al. (2011) Neurobiology of disease 41:706-716). This phenomenon was more pronounced in the corrected cell line, suggesting increased mitochondrial MPT pore opening threshold levels in this cell line.

### Example 5

### Spontaneous Caspase Activation is Rescued by ZFN Correction of the LRRK2 G2019S Mutation

Next, we investigated if the observed differences between the ZFN-corrected and LRRK2 G2019S NPCs also resulted in differential levels of cellular apoptosis activation through caspases. See, e.g., Cookson (2012) Biochemical Society transactions 40:1070-1073. We therefore tested caspase 3/7 activation in live cells that were stressed by rotenone and nutritional stress. We detected differences in spontaneous caspase activation between the two cell lines even in the absence of apoptosis inducers with an increase caspase 3/7 level in the LRRK2 mutations whereas we showed less activation in the ZFN-corrected line (FIG. 4D).

In summary, these data suggest that correction of the LRRK2 phenotype improves viability and cell survival capabilities of NPCs even under normal growth conditions. These data also suggest that the susceptibility to oxidative stress and subsequently increase ROS could lead the additional mitochondrial damage at the mitochondrial DNA level.

### Example 6

### Gene Correction of LRRK2 G2019S Mutation Reverses Mitochondrial DNA Damage in iPSC-Derived Neural Cells from Parkinson's Disease Patients

In order to study mitochondrial DNA damage in a neuronal (PD) context, we applied cellular reprogramming technology to determine changes caused by mutations of LRRK2. Given the mitochondrial deficits of iPSC-derived neural cells from subjects carrying LRRK2 mutations and the fact that mtDNA damage compromises mitochondrial and neuronal function, we carried out experiments to determine if LRRK2 PD iPSC-derived neural cells exhibit mtDNA damage. Cooper et al. (2012) Sci Transl Med 4:141ra90 (2012).

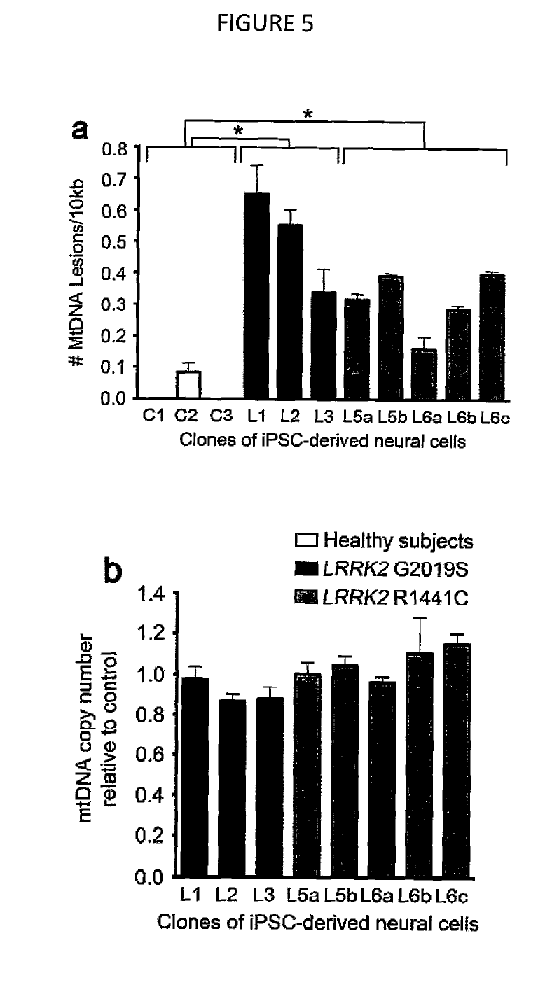

iPSCs were derived from three patients carrying the homozygous or heterozygous LRRK2 G2019S mutation, two asymptomatic subjects carrying the heterozygous LRRK2 R1441C mutation, and three age-matched healthy subjects without LRRK2 mutations. Multiple iPSC clones were examined from each individual carrying the LRRK2 R1441C mutation. Two differentiation protocols were used to generate cells for analysis of mtDNA damage. The analysis of mtDNA damage across neural cells from multiple patients and healthy subjects (FIG. 5) used a differentiation protocol that had previously been used to determine mitochondrial deficits in neural cells. Cooper et al. (2012) Sci Transl Med 4:141ra90 (2012). Mak et al. (2012). Stem Cells Int 2012, 140427.

The differentiation of immature neuroprogenitor cells and more mature neural cells from repaired iPSCs for analyses of mtDNA damage was performed as described. MtDNA damage was increased in iPSC-derived cells carrying LRRK2 mutations differentiated with either protocol.

Upon neuronal differentiation of the iPSC lines, cultures were harvested, pelleted and coded for blinded analysis. After receipt of the coded samples, DNA was purified and a quantitative polymerase chain reaction (QPCR)-based assay specific for the mitochondrial genome determined mtDNA damage. This method is based on the principle that various forms of DNA damage have the propensity to slow down or block DNA polymerase progression. Santos, et al. (2006) Methods Mol Biol 314:183-99. Thus, if equal amounts of mtDNA from experimental and control specimens are amplified under identical conditions, the mtDNA sample with the least mtDNA damage will produce the greatest amount of PCR product.

Using this approach, as shown in FIG. 5, a significant increase in levels of mtDNA damage was found in neural cells derived from individuals carrying either the homozygous or heterozygous LRRK2 G2019S (black bars, p<0.0001 ANOVA) or heterozygous R1441C (grey bars, p<0.0001 ANOVA) mutations compared to neural cells from healthy subjects (white bars, FIG. 5A). Mitochondrial DNA copy number was similar across all clones (FIG. 5B).

While increased levels of mtDNA damage in LRRK2 neural cells were observed across the different pathogenic mutations, across multiple clones from single individuals and in siblings carrying the same mutation (FIG. 5), we used two additional approaches to further strenghten our interpretation that LRRK2 mutations induce mtDNA damage. First, we measured mtDNA damage in iPSC-derived neuroprogenitor cells (NPCs) from two brothers. NPCs from a PD patient carrying the heterozygous LRRK2 G2019S mutation (iPSC clone L4a) showed greater levels of mtDNA damage compared to his healthy brother who did not carry the LRRK2 mutation.

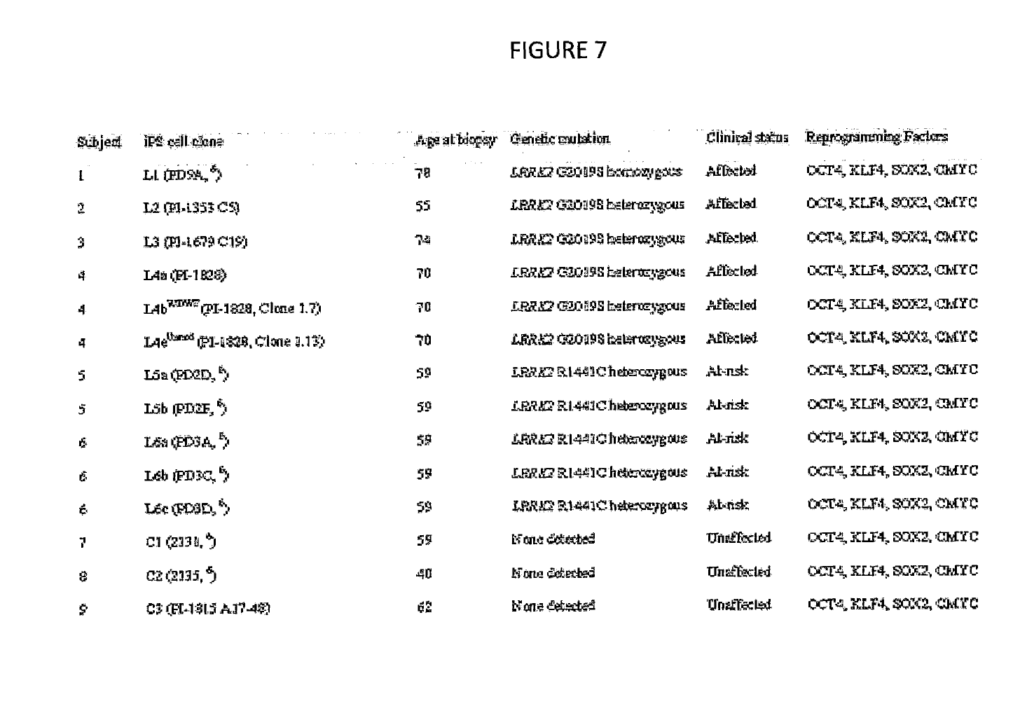

Second, we addressed the issue of genetic and biological variability between patient and healthy subject control iPSCs. Ideally, isogenic cell lines that differ from the original culture lines only by a disease-causing mutation should be used for study. Without such isogenic lines, there may be difficulties in data interpretation, because personal genomic variation may cause functionally relevant differences between individuals. We therefore used zinc finger nucleases (ZFNs) to repair the LRRK2 G2019S mutation (iPSC clone L4b^{WT/WT}, FIG. 7). In parallel, we compared our assays with an iPSC clone from the same parental iPSC line (iPSC clone L4a) that was not modified during the ZFN process and had retained the LRRK2 G2019S mutation (unmodified mutant iPSC clone L4e^{Unmod}, FIG. 7). To examine levels of mtDNA damage, we differentiated repaired and control unmodified LRRK2 G2019S iPSC clones into neuroprogenitor cells (NPCs) and neural cells. Mak et al. (2012). Stem Cells Int 2012, 140427.

Immunocytochemistry revealed that the control and repaired iPSC clones differentiated into NPCs that expressed nestin and SOX1 (FIGS. 6A and B), and neural cells that included dopaminergic neurons expressing tyrosine hydroxylase (TH) and β-III tubulin (FIGS. 6D and 6E). NPCs and neural cells differentiated from the repaired LRRK2 G2019S iPSCs showed less mtDNA damage than similar cells from the control unmodified iPSCs (p<0.0001 and p<0.002, respectively; FIGS. 6C and 6F). The number of mtDNA genomes in neural cells and NPCs differentiated from repaired and control LRRK2 G2019S iPSCs was similar.

The mechanisms that result in mitochondrial dysfunction in PD include intrinsically high levels of reactive oxygen species (ROS) (Guzman et at (2010). Nature 468:696-700) and environmental factors that can cause significant oxidative damage and neurodegeneration. See, e.g., Langston et al. (1983) Science 219, 979-980 (1983); Betarbet et al. (2000) Nat Neurosci 3:1301-6.

Thus, LRRK2 mutations are associated with mtDNA damage, even in neural cells derived from presymptomatic mutation carriers. Thus, mtDNA damage could be an early event in the pathogenesis of PD. The use of ZFNs to repair the genetic mutation in otherwise isogenic cells abrogated the mitochondrial phenotype, thereby providing strong evidence that LRRK2 mutations cause mtDNA damage in neural cells. Because LRRK2 mutations are a common cause of both sporadic and autosomal dominant PD, examination of the molecular targets of LRRK2 and their potential roles in mtDNA damage is likely to provide critical mechanistic and therapeutic insights into both LRRK2-related parkinsonism and idiopathic PD. The QPCR-based assay we used simultaneously detects a wide variety of types of mtDNA damage, including strand breaks, apurinic/apyrimidinic sites, modified purines and pyridines and DNA repair intermediates. Future studies to identify the specific types of mtDNA damage in mutant LRRK2 neural cells will help identify the critical DNA repair pathways involved and may suggest additional therapeutic targets.

Our data demonstrate that mtDNA damage is induced in neural cells by PD-associated mutations in LRRK2, and this phenotype can be functionally reversed by ZFN-mediated genome editing in iPSCs. These results indicate that mtDNA damage could be a critical event in neuronal dysfunction that contributes to the highly variable penetrance and onset of LRRK2 mutations causing PD between 40-94 years of age.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of determining whether a compound is useful in the treatment of Parkinson's disease or Parkinson's-related disease, the method comprising:
(a) providing an isogenic cell line comprising a modified LRRK2 allele;
(b) contacting the isogenic cell line with the compound under conditions of oxidative stress; and
(c) assaying the isogenic cell line for a response to the compound, thereby screening a compound for reducing sensitivity and/or response to oxidative stress.

2. A method of determining whether a compound is useful in the treatment of Parkinson's disease or Parkinson's-related disease, the method comprising:
(a) providing an isogenic cell line comprising a modified LRRK2 allele;
(b) contacting the isogenic cell line with the compound; and
(c) assaying the isogenic cell line for a reduction of mitochondrial DNA damage or a reduction in the rate of mitochondrial DNA damage, thereby determining whether the agent is useful in the treatment of Parkinson's disease or Parkinson's-related disease.

3. The method of claim 2, further comprising comparing the isogenic cell line to a control cell line.

4. The method of claim 3, wherein the amount of free radicals produced by the control cell is measured in the absence of the compound.

5. The method of claim 1, wherein the response is production of free radicals, mitochondrial membrane potential (MMP), mitochondrial transitional pore opening (MTP) or caspase activation.

6. The method of claim 5, wherein the response is production of free radicals and the compound reduced the amount of free radicals in the isogenic cell line.

7. The method of claim 5, wherein the response is MMP and the compound increases the MMP.

8. The method of claim 5, wherein the response is MTP and the compound increases the MTP.

9. The method of claim 5, wherein the MTP is measured by loading with a calcium chelator.

10. The method of claim 9, the calcium chelator is calcein.

11. The method of claim 5, wherein the response is caspase activation and the caspase activation is decreased.

12. The method of claim 1, the condition of oxidative stress is selected from the group consisting of nutrient withdrawal, presence of a toxin and combinations thereof.

13. The method of claim 12, wherein the condition of oxidative stress is the presence of a toxin and the toxin is rotenone or staurosporine.

14. The method of claim 1, wherein the modified LRRK2 allele comprises a G2019S mutation.

15. The method of claim 2, wherein the modified LRRK2 allele comprises one or more mutations.

16. The method of claim 15, wherein the modified LRRK2 allele comprises a G2019S mutation.

17. A method of evaluating the prognosis or severity of Parkinson's disease or Parkinson's-related disease in a subject, the method comprising:
(a) isolating a sample from a subject identified to carry a mutation in a gene associated with Parkinson's disease or Parkinson's-related disease;
(b) assaying the level of mitochondrial DNA damage in the sample;
(c) determining the prognosis or severity of Parkinson's disease or Parkinson's-related disease based on the level of mitochondrial DNA damage in the sample, where a high level of mitochondrial DNA correlates to a more severe grade of Parkinson's disease or Parkinson's-related disease or to earlier onset of Parkinson's disease or Parkinson's-related disease.

18. The method of claim 17, wherein the gene is LRRK2.

19. The method of claim 18, wherein the LRRK2 gene comprises a G2019S mutation.

20. The method of claim 17, wherein the subject is undergoing therapy for Parkinson's disease or Parkinson's-related disease.
